(19) United States
(12) Patent Application Publication
(10) Pub. No.: US 2023/0165771 A1
(43) Pub. Date: Jun. 1, 2023

(54) PROCESS FOR TREATING KERATIN FIBERS USING PARTICULAR AMINO ACIDS IN HIGH CONCENTRATION

(71) Applicant: L'Oreal, Paris (FR)

(72) Inventors: Leila HERCOUET, SAINT-OUEN (FR); Henri SAMAIN, CHEVILLY LARUE (FR); Simon DONCK, SAINT-OUEN (FR); Christian BLAISE, AULNAY-SOUS-BOIS (FR)

(21) Appl. No.: 18/013,657

(22) PCT Filed: Jun. 30, 2021
(86) PCT No.: PCT/EP2021/068034

§ 371 Date: Dec. 29, 2022

(30) Foreign Application Priority Data
Jun. 30, 2020 (FR) FR2006869
Dec. 11, 2020 (FR) FR2013092

(51) Int. Cl. A61K 8/44; A61Q 5/08; A61Q 5/10; A61Q 5/12

(52) U.S. Cl. CPC A61K 8/44; A61Q 5/08; A61Q 5/10; A61Q 5/12; A61K 2800/884; A61K 2800/4324

(57) ABSTRACT

The present invention relates to a process for treating keratin fibers, comprising a step of applying to the keratin fibers a composition comprising a high content of one or more particular amino acids followed by a step of permanent reshaping of the keratin fibers.

### FIELD OF THE INVENTION

The present invention relates to a process for treating keratin fibers, comprising a step of applying to the keratin fibers a composition comprising a high content of one or more particular amino acids followed by a step of permanent reshaping of the keratin fibers.

### BACKGROUND OF THE INVENTION

Many people are unsatisfied with the appearance of their hair: in particular, people who have curly hair usually wish to obtain straight hair, and, conversely, people who have curl-free hair wish to have curly hair.

Several techniques exist for permanently modifying the shape of the hair. A first technique consists in performing a “lanthionization” operation, using a composition comprising a base belonging to the hydroxide family. It leads to replacement of the disulfide bonds (—CH₂—S—S—CH₂—), also known as disulfide bridges, present in keratin with lanthionine bonds (—CH₂—S—CH₂—). It is performed in a single step and is mainly used for shaping naturally frizzy hair.

A second technique consists, in a first stage, in opening the disulfide covalent bonds, generally by means of a basic composition comprising a thiol-based reducing agent (reduction step), and then, after having rinsed the head of hair thus treated, generally with water, in reconstituting, in a second stage, said disulfide bonds by applying to the hair, which has been placed under tension beforehand, an oxidizing composition (oxidation step, also known as the fixing step) so as finally to give the hair the desired shape. A major drawback of these various techniques is that they may lead to a reduction in the cosmetic properties of the hair, such as a loss of vitality and sheen, and degradation of its mechanical properties, notably its mechanical strength, in particular when the treated hair is hair that is already embrittled, for instance dyed or bleached hair. This degradation of the mechanical strength of the hair may lead to substantial breakage during combing or blow-drying of the hair. Thus, it is common practice to resort to care compositions involving conditioning agents in order to limit the degradation or to improve the quality of the keratin fibers. However, these care compositions may, in certain cases, result in lower efficiency of the shaping treatment.

There is thus a real need to develop a process for treating keratin fibers which makes it possible to conserve or even to improve the quality of the keratin fibers, including sensitized hair, notably dyed or bleached hair, and to reduce the breakage thereof, while at the same time enabling efficient permanent reshaping of the keratin fibers. Such a process must also involve compositions that are easy to use, notably easy to apply and stable and that make it possible to maintain or even to improve the cosmetic properties of the keratin fibers thus treated, for example as regards the sheen, the soft feel, the suppleness, the appearance or the disentangling. Furthermore, such a process will ideally need to be compatible with any type of permanent reshaping composition that is commercially available.

The Applicant has discovered, surprisingly, that all of these objectives can be achieved by means of the process according to the present invention.

### SUMMARY OF THE INVENTION

According to a first aspect, a subject of the present invention is a process for treating keratin fibers, comprising the following successive steps:

i) a step of applying to the keratin fibers a composition (A) comprising one or more amino acids chosen from the compounds of formula (I₁) salts thereof and mixtures thereof:

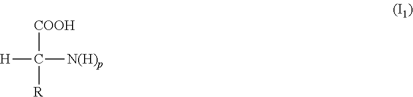

in which formula (I₁):
p is an integer equal to 1 or 2;
when p=1, R forms with the nitrogen atom a saturated 5- to 8-membered, preferably 5-membered, heterocycle, this ring possibly being optionally substituted with at least one group chosen from hydroxyl or (C₁-C₄)alkyl;
when p=2, R represents:
a hydrogen atom; or
a (C₁-C₁₂)alkyl group, preferably a (C₁-C₄)alkyl group, interrupted with at least one heteroatom or group chosen from —S—, —NH— or —C(NH)— and/or substituted with at least one group chosen from hydroxyl, amino or —NH—C(NH)—NH₂;

the amino acid(s) being present in composition (A) in a total content of at least 5% by weight relative to the total weight of composition (A);

ii) a step of permanent reshaping of the keratin fibers.

According to a second aspect, a subject of the present invention is composition (A) as defined previously.

According to a third aspect, a subject of the present invention is the use of a composition (A) as defined previously, as a pretreatment composition of a process for permanently reshaping keratin fibers.

According to a fourth aspect, a subject of the present invention is the use of a composition (A) as defined previously, for protecting keratin fibers, preferably for protecting them against breakage, during a treatment for permanently reshaping the keratin fibers.

According to a fifth aspect, a subject of the present invention is a multi-compartment device comprising:
a first compartment containing a composition (A) as defined previously; and
a second compartment containing a composition comprising at least one thiol-based or non-thiol reducing agent or at least one alkaline agent chosen from mineral hydroxides, organic hydroxides and mixtures thereof; and
optionally a third compartment containing a composition comprising at least one chemical oxidizing agent; and
optionally a fourth compartment containing a composition comprising at least one coloring agent chosen from oxidation dyes, direct dyes and mixtures thereof.

### DETAILED DESCRIPTION OF THE INVENTION

For the purposes of the present invention and unless otherwise indicated:
the term “keratin fibers” means fibers of human or animal origin, such as head hair, bodily hair, the eyelashes, the eyebrows, wool, angora, cashmere or fur. According to the present invention, the keratin fibers are preferably human keratin fibers, more preferentially the hair.
the term “successive steps” means steps performed in the indicated order.
the term “permanently reshaping keratin fibers” means the permanent-waving, straightening or relaxing of keratin fibers.
the term “dye composition” means a composition comprising at least one coloring agent.
the term “bleaching composition” means a composition comprising at least one chemical oxidizing agent.
the term “alkyl group” means a linear or branched, saturated hydrocarbon-based radical.
the term “(Cₓ-C_{y})alkyl group” means an alkyl group comprising from x to y carbon atoms.
the term “coloring agent” means an oxidation dye, a direct dye or a pigment.
the term “oxidation dye” means an oxidation dye precursor chosen from oxidation bases and couplers. Oxidation bases and couplers are colorless or sparingly colored compounds, which, via a condensation reaction in the presence of an oxidizing agent, give a colored species.
the term “direct dye” means a natural and/or synthetic dye, including in the form of an extract or extracts, other than oxidation dyes. These are colored compounds that will spread superficially on the fiber. They may be ionic or nonionic, i.e. anionic, cationic, neutral or nonionic.
the term “reducing agent” means an agent that is capable of reducing the disulfide bonds of the hair, such as compounds chosen from thiols, alkaline sulfites, hydrides and phosphines.
the term “chemical oxidizing agent” means an oxidizing agent other than atmospheric oxygen.

Unless otherwise indicated, when compounds are mentioned in the present patent application, this also includes the optical isomers thereof, the geometrical isomers thereof, the tautomers thereof, the salts thereof or the solvates thereof, alone or as a mixture.

The expressions “at least one” and “one or more” are synonymous and may be used interchangeably.

Process for Treating Keratin Fibers

According to a first aspect, a subject of the present invention is a process for treating keratin fibers as defined previously.

The Applicant has found, surprisingly, that the process according to the invention makes it possible to conserve or even to improve the quality of the keratin fibers, including sensitized hair, notably dyed or bleached hair, and to reduce the breakage thereof, while at the same time enabling efficient permanent reshaping of the keratin fibers. Furthermore, the process according to the invention involves a pretreatment step which is different from the step of permanent reshaping of the keratin fibers and is thus compatible with any type of permanent reshaping composition available on the market.

In the process according to the present invention, it is essential for steps i) and ii) to be successive, i.e. for step ii) to be performed after step i).

The process may comprise one or more additional steps between steps i) and ii), but, even in such an embodiment, step ii) is always performed after step i).

Composition (A)

Amino Acids

Composition (A) applied to the keratin fibers during step i) of the process comprises one or more amino acids chosen from the compounds of formula (I₁) as defined previously, salts thereof and mixtures thereof, preferably chosen from the compounds of formula (I₁).

The salts of compounds of formula (I₁) comprise the salts with organic or mineral bases, for example the salts of alkali metals, for instance the lithium, sodium or potassium salts; the salts of alkaline-earth metals, for instance the magnesium or calcium salts, and the zinc salts.

The compounds of formula (I₁) may be in the form of an optical isomer of L, D or DL configuration, preferably of L configuration.

As examples according to the present invention of compounds of formula (I₁) in the form of an optical isomer of L configuration, mention may be made of L-proline, L-methionine, L-serine, L-arginine and L-lysine.

Preferably, the amino acid(s) included in composition (A) are chosen from glycine, proline, methionine, serine, arginine, lysine, salts thereof and mixtures thereof.

More preferentially, the amino acid(s) included in composition (A) are chosen from glycine, proline, methionine, serine, salts thereof and mixtures thereof.

Even more preferentially, the amino acid included in composition (A) is chosen from glycine, salts thereof and mixtures thereof.

As examples of glycine salts that may be used in the present invention mention may be made of sodium glycinate, zinc glycinate, calcium glycinate, magnesium glycinate, manganese glycinate and potassium glycinate, preferably sodium glycinate or potassium glycinate.

Particularly preferably, the amino acid included in composition (A) is glycine.

The amino acid(s) chosen from the compounds of formula (I₁), salts thereof and mixtures thereof are present in composition (A) in a total content of at least 5% by weight, preferably of at least 8% by weight, relative to the total weight of composition (A).

The amino acid(s) chosen from the compounds of formula (I₁), salts thereof and mixtures thereof may be present in composition (A) in a total content ranging from 5% to 20% by weight, preferably ranging from 5% to 15% by weight, more preferentially ranging from 8% to 12% by weight, relative to the total weight of composition (A).

Composition (A) may preferably comprise at least 5% by weight, more preferentially at least 8% by weight, of glycine, salts thereof and mixtures thereof relative to the total weight of composition (A).

Preferably, composition (A) comprises from 5% to 20% by weight, preferably from 5% to 15% by weight and more preferentially from 8% to 12% by weight of glycine, salts thereof and mixtures thereof relative to the total weight of composition (A).

According to a preferred embodiment, composition (A) applied to the keratin fibers during step i) of the process comprises one or more amino acids chosen from the compounds of formula (I₁) as defined previously.

According to this preferred embodiment, the amino acid(s) included in composition (A) are preferably chosen from glycine, proline, methionine, serine, arginine, lysine and mixtures thereof, more preferentially from glycine, proline, methionine, serine and mixtures thereof, and, even more preferentially, the amino acid included in composition (A) is glycine.

According to this preferred embodiment, the amino acid(s) chosen from the compounds of formula (I₁) are present in composition (A) in a total content of at least 5% by weight, preferably of at least 8% by weight, relative to the total weight of composition (A).

According to this preferred embodiment, the amino acid(s) chosen from the compounds of formula (I₁) are present in composition (A) in a total content preferably ranging from 5% to 20% by weight, more preferentially ranging from 5% to 15% by weight, even more preferentially ranging from 8% to 12% by weight, relative to the total weight of composition (A).

According to this preferred embodiment, composition (A) may preferably comprise at least 5% by weight, more preferentially at least 8% by weight, of glycine relative to the total weight of composition (A).

According to this preferred embodiment, composition (A) preferably comprises from 5% to 20% by weight, more preferentially from 5% to 15% by weight and even more preferentially from 8% to 12% by weight of glycine relative to the total weight of composition (A).

pH

Composition (A) may have a pH ranging from 2 to 11. Preferably, the pH of composition (A) ranges from 4 to 10. More preferentially, the pH of composition (A) ranges from 8 to 10. By way of example, the pH of composition (A) may be equal to 9.

According to one embodiment, the pH of composition (A) ranges from 2 to 4.

The pH of composition (A) may be adjusted with at least one organic or mineral acid, or with at least one alkaline agent chosen from mineral or organic or hybrid alkaline agents and mixtures thereof.

The term “organic acid” means an acid, i.e. a compound that is capable of releasing a cation or proton H⁺ or H₃O⁺, in aqueous medium, which includes at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfonic SO₃H, sulfinic SO₂H, phosphonic POSH and phosphinic PO₂H₂.

More particularly, the organic or mineral acid used is chosen from hydrochloric acid HCl, hydrobromic acid HBr, sulfuric acid H₂SO₄, alkylsulfonic acids: (C₁-C₆)Alk-S(O)₂O H such as methylsulfonic acid and ethylsulfonic acid; arylsulfonic acids: Ar—S(O)₂OH such as benzenesulfonic acid and toluenesulfonic acid; (C₁-C₆)alkoxysulfinic acids: Alk-O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H and tetrafluoroboric acid HBF₄, and carboxylic acid(s) of formula (II) below and the salts thereof:

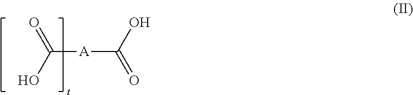

in which formula (II):

A represents a saturated or unsaturated, cyclic or noncyclic, aromatic or nonaromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted, notably with one or more hydroxyl groups; preferably, A represents a monovalent (C₁-C₆)alkyl group or a polyvalent (C₁-C₆)alkylene group optionally substituted with one or more hydroxyl groups.

In particular, the acid used is chosen from the carboxylic acids of formula (II) as defined previously. Preferably, the acid used is an α-hydroxy acid such as lactic acid, glycolic acid, tartaric acid or citric acid.

The mineral alkaline agents are preferably chosen from aqueous ammonia, alkaline carbonates or bicarbonates such as sodium or potassium carbonates and sodium or potassium bicarbonates, sodium hydroxide or potassium hydroxide, and mixtures thereof.

The organic alkaline agents are preferably chosen from organic amines, i.e. they contain at least one substituted or unsubstituted amino group.

The organic alkaline agents are more preferentially chosen from organic amines with a pK_{b} at 25° C. of less than 12, preferably of less than 10 and even more advantageously of less than 6. It should be noted that it is the pK_{b} corresponding to the function which has the highest basicity.

The organic alkaline agents are chosen, for example, from alkanolamines, oxyethylenated and/or oxypropylenated ethylenediamines and the compounds of formula (III) below:

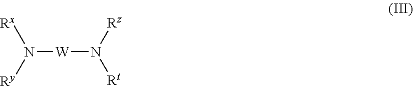

in which formula (III):
W is a divalent C₁-C₆ alkylene group optionally substituted with a hydroxyl group or a (C₁-C₆)alkyl group, and/or optionally interrupted with one or more heteroatoms such as oxygen or NRu;
R^{x}, R^{y}, R^{z} R^{t} and R^{u}, which may be identical or different, represent a hydrogen atom or a group chosen from (C₁-C₆)alkyl, C₁-C₆ hydroxyalkyl or C₁-C₆ aminoalkyl.

Preferably, the alkanolamine is ethanolamine (or monoethanolamine).

In one variant of the invention, composition (A) comprises, as alkaline agent, one or more alkanolamines (preferably ethanolamine) and aqueous ammonia. In this variant, the alkanolamine(s) are present in a predominant amount relative to the aqueous ammonia.

Hybrid alkaline agents that may be mentioned include the salts of the amines mentioned previously with acids such as carbonic acid or hydrochloric acid.

Cationic Polymers

Composition (A) may comprise one or more cationic polymers.

The term “cationic polymer” means any polymer comprising cationic groups and/or groups that can be ionized into cationic groups and not comprising any anionic groups and/or groups that can be ionized into anionic groups. Preferably, the cationic polymer is hydrophilic or amphiphilic.

The preferred cationic polymers are chosen from those that contain units including primary, secondary, tertiary and/or quaternary amine groups that may either form part of the main polymer chain or may be borne by a side substituent directly connected thereto. The cationic polymers that may be used preferably have a weight-average molar mass (Mw) ranging from 500 to 5×10⁶ g/mol and preferably ranging from 10³ to 3×10⁶ g/mol.

Preferably, composition (A) comprises one or more cationic polymers chosen from homopolymers or copolymers including in their structure one or more units corresponding to formula (I) or (II):

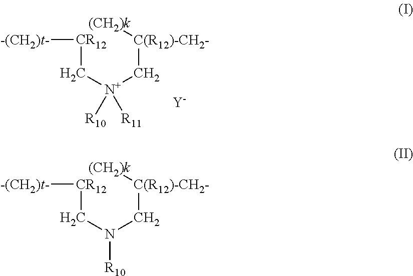

in which:
k and t are equal to 0 or 1, the sum k+t being equal to 1;
R₁₂ denotes a hydrogen atom or a methyl group;
R₁₀ and R₁₁, independently of each other, denote a (C₁-C₆)alkyl group, a C₁-C₅ hydroxyalkyl group, a C₁-C₄ amidoalkyl group; or alternatively R₁₀ and R₁₁ denote, together with the nitrogen atom to which they are attached, a heterocyclic group such as piperidyl or morpholinyl; preferably, R₁₀ and R₁₁, independently of each other, denote a (C₁-C₄)alkyl group;
Y is an anion preferably chosen from bromide, chloride, acetate, borate, citrate, tartrate, bisulfate, bisulfite, sulfate and phosphate.

More preferentially, composition (A) comprises one or more cationic polymers chosen from homopolymers or copolymers including in their structure one or more units corresponding to formula (I) as defined previously.

Even more preferentially, composition (A) comprises one or more cationic polymers chosen from homopolymers of diallyldimethylammonium salts and copolymers of diallyldimethylammonium salts and of acrylamide.

Particularly preferably, composition (A) comprises one or more cationic polymers chosen from copolymers of diallyldimethylammonium salts and of acrylamide.

Mention may be made more particularly of the homopolymer of dimethyldiallylammonium salts (for example chloride) for example sold under the name Merquat 100 by the company Nalco and the copolymers of diallyldimethylammonium salts (for example chloride) and of acrylamide, notably sold under the name Merquat 550 or Merquat 7SPR.

The cationic polymer(s) may be present composition (A) in a total content ranging from 0.00001% to 5% by weight, preferably ranging from 0.00005% to 1% by weight and more preferentially ranging from 0.00007% to 0.5% by weight, relative to the total weight of composition (A).

Amino Silicones

Composition (A) may comprise one or more silicones, preferably chosen from amino silicones.

The term “amino silicone” means any silicone including at least one primary, secondary or tertiary amine function.

The weight-average molecular masses of these amino silicones may be measured by gel permeation chromatography (GPC) at room temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Preferably, the amino silicones are chosen from the amino silicones of formula (B) below:

R′ₐG₃₋ₐ—Si(OSiG₂)ₙ—(OSiG_{b}R′_{2-b})ₘ—O-SiG₃₋ₐ—R′ₐ (B)

in which:
G, which may be identical or different, denotes a hydrogen atom or a group from among phenyl, OH, C₁-C₈ alkyl, for example methyl, or C₁-C₈ alkoxy, for example methoxy,
a, which may be identical or different, denotes 0 or an integer from 1 to 3, in particular 0,
b denotes 0 or 1, in particular 1,
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10,
R′, which may be identical or different, denotes a monovalent radical of formula —CqH2qL in which q is a number ranging from 2 to 8 and L is an optionally quaternized amine group chosen from the following groups:

—N(R″)₂; —N⁺(R″)₃ A-; —NR″-Q-N(R″)₂ and —NR″-Q-N⁺(R″)₃ A-,

in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, notably a halide such as fluoride, chloride, bromide or iodide.

More preferentially, the amino silicones are chosen from the amino silicones of formula (F) below:

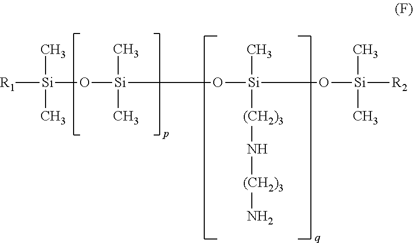

in which:
p and q are numbers such that the sum (p+q) ranges from 1 to 1000, in particular from 50 to 350 and more particularly from 150 to 250; p possibly denoting a number from 0 to 999, notably from 49 to 349 and more particularly from 159 to 239, and q possibly denoting a number from 1 to 1000, notably from 1 to 10 and more particularly from 1 to 5;
R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000, even more particularly from 5000 to 100 000 and more particularly from 10 000 to 50 000.

The commercial products comprising silicones of structure (F) may include in their composition one or more other amino silicones whose structure is different from that of formula (F).

A product containing amino silicones of structure (F) is sold by Wacker under the name Fluid WR 13000.

Among the amino silicones of formula (F), mention may also be made of the product Belsil ADM Log 1 from Wacker.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometers.

The silicone(s) may be present in composition (A) in a total content ranging from 0.001% to 10% by weight, preferably ranging from 0.01% to 5% by weight, more preferentially ranging from 0.02% to 1% by weight, even more preferentially ranging from 0.05% to 0.5% by weight relative to the total weight of composition (A).

The amino silicone(s) may be present composition (A) in a total content ranging from 0.001% to 10% by weight, preferably ranging from 0.01% to 5% by weight, more preferentially from 0.02% to 1% by weight and even more preferentially ranging from 0.05% to 0.5% by weight, relative to the total weight of composition (A).

Composition (A) preferably comprises a total content of coloring agents and/or reducing agents of less than 0.1% by weight, more preferentially less than 0.01% by weight, even more preferentially less than 0.001% by weight, relative to the total weight of composition (A).

According to a particularly preferred embodiment, composition (A) is free of coloring agents and/or reducing agents.

Composition (A) preferably comprises a total content of chemical oxidizing agents of less than 0.1% by weight, more preferentially less than 0.01% by weight, even more preferentially less than 0.001% by weight, relative to the total weight of composition (A).

Surfactants

Composition (A) preferably comprises less than 5% by weight, more preferentially less than 2% by weight and even more preferentially less than 1% by weight of surfactants relative to the total weight of the composition.

In particular, composition (A) may comprise a total content of anionic surfactants of less than 0.1% by weight, preferably less than 0.01% by weight, more preferentially less than 0.001% by weight, relative to the total weight of composition (A).

According to a particularly preferred embodiment, composition (A) is free of anionic surfactants.

Composition (A) may comprise a total content of nonionic surfactants of less than 0.5% by weight relative to the total weight of composition (A).

Water

Composition (A) may comprise a total content of water ranging from 1% to 95% by weight, preferably ranging from 20% to 95%, more preferentially ranging from 40% to 90% by weight and even more preferentially ranging from 60% to 85% by weight, relative to the total weight of composition (A).

Organic Solvents

Composition (A) may comprise at least one organic solvent, preferably chosen from monoalcohols, polyols, polyol ethers and mixtures thereof.

Composition (A) may comprise at least 5% by weight and preferably at least 8% by weight, relative to the total weight of composition (A), of one or more monoalcohols. The monoalcohols may be linear or branched.

The monoalcohols are preferably chosen from C₂ to C₆ monoalcohols, more preferentially from C₂ to C₄ monoalcohols, even more preferentially from ethanol, isopropanol, tert-butanol, n-butanol and mixtures thereof.

According to a particularly preferred embodiment, the monoalcohol is ethanol.

The monoalcohol(s) may be present in composition (A) in a total content ranging from 5% to 20% by weight, preferably ranging from 5% to 15% by weight, more preferentially ranging from 8% to 12% by weight, relative to the total weight of composition (A).

The polyols are preferably chosen from propylene glycol, dipropylene glycol, glycerol and mixtures thereof.

The polyol ethers are preferably chosen from propylene glycol monomethyl ether, diethylene glycol monomethyl ether and monoethyl ether and mixtures thereof.

Composition (A) may comprise a total content of organic solvents ranging from 1% to 40% by weight, preferably ranging from 5% to 30% by weight, more preferentially ranging from 8% to 15% by weight, relative to the total weight of the composition.

Step of Permanent Reshaping of the Keratin Fibers

The process comprises a step ii) of permanent reshaping of keratin fibers. Step ii) of permanent reshaping of keratin fibers is preferably a step of straightening or relaxing keratin fibers.

Reducing Agents

The step of permanent reshaping of keratin fibers may comprise the application to the keratin fibers of a composition comprising one or more thiol-based or non-thiol reducing agents, preferably one or more thiol-based reducing agents.

Thiol-Based Reducing Agents

The thiol-based reducing agent(s) are chosen from organic compounds including one or more —SH, —S— or disulfide (—S—S—) groups, preferably —SH groups, and at least one other function chosen from carboxylic acid, amine, amide, ester and alcohol functions and mixtures thereof.

According to a particular embodiment of the invention, the thiol-based reducing agent(s) are chosen from those of formulae i-1 and i-2, and mixtures thereof:

R₁—SH i-1

R′—S—R″ i-2

in which formulae i-1 and i-2:
R₁ represents:
a (C₁-C₈)alkyl and preferably (C₁-C₆)alkyl group, optionally substituted with one or more groups chosen from carboxyl C(O)OH, (di)(C₁-C₄)(alkyl)amino, hydroxyl —OH, thiol —SH or —C(O)—NH—CH₂—C(O)OH and/or optionally interrupted with one or more heteroatoms or groups chosen from 0, S, C(O) or combinations thereof such as —O—C(O)—, —C(O)—O—, —N(R′″)—C(O)—, or —C(O)—N(R′″)—; with R′″ representing a hydrogen atom or a (C₁-C₆)alkyl group; or
a (hetero)aryl group optionally substituted with one or more hydroxyl, thiol or carboxyl groups;
R′ and R″, which may be identical or different, represent a (C₁-C₈)alkyl and preferably (C₁-C₆)alkyl group, substituted with one or more groups chosen from hydroxyl, thiol and carboxyl;

or else R′ and R″ form, together with the sulfur atom which bears them, a 5- to 7-membered heterocyclic group, which is preferably saturated, which comprises from 1 to 3 heteroatoms, and which is optionally substituted with one or more (C₁-C₆)alkyl groups optionally substituted with one or more hydroxyl, thiol or carboxyl groups; more preferentially, the heterocyclic group is a dithiolane group optionally substituted with a (C₁-C₆)alkyl group optionally substituted with one or more carboxyl groups.

According to a particular embodiment of the invention, the thiol-based reducing agent(s) are chosen from those of formula i-1, in particular from those of formula i-1 in which R₁ represents a (C₁-C₈)alkyl and preferably (C₁-C₆)alkyl group,
substituted with one or more groups chosen from carboxyl C(O)OH, amino, hydroxyl —OH and thiol —SH; and/or
optionally interrupted with one or more heteroatoms or groups chosen from —O—, —N(R′″)—, C(O) or combinations thereof such as —O—C(O)—, —C(O)—O—, —N(R′″)—C(O)—, or —C(O)—N(R′″)—, with R′″ representing a hydrogen atom or a (C₁-C₆)alkyl group.

According to a preferred embodiment of the invention, the thiol-based reducing agent(s) are chosen from those of formula i-1 in which R₁ represents a (C₁-C₈)alkyl and preferably (C₁-C₆)alkyl group, which is not interrupted and substituted with one or more groups chosen from carboxyl C(O)OH, amino, hydroxyl —OH, and thiol —SH.

According to another particular embodiment of the invention, the thiol-based reducing agent(s) are chosen from those of formula i-1 in which R₁ represents:
a phenyl group optionally substituted with one or more groups chosen from carboxyl C(O)OH, amino, hydroxyl —OH, and thiol —SH; or
a 5- to 10-membered, preferably 9- or 10-membered bicyclic, heteroaryl group, comprising from 1 to 4 heteroatoms chosen from O, S or N, preferably N, optionally substituted with one or more hydroxyl or thiol groups.

According to another particular embodiment of the invention, the thiol-based reducing agent(s) are chosen from those of formula i-2 in which R′ and R″, which may be identical or different represent a (C₁-C₈)alkyl and preferably (C₁-C₆)alkyl group, substituted with one or more groups chosen from hydroxyl, thiol and carboxyl.

According to another particular embodiment of the invention, the thiol-based reducing agent(s) are chosen from those of formula i-2 in which R′ and R″ form, together with the sulfur atom which bears them, a 5- to 7-membered heterocyclic group, which is preferably saturated, which comprises from 1 to 3 heteroatoms, and which is optionally substituted with one or more (C₁-C₆)alkyl groups optionally substituted with one or more hydroxyl, thiol or carboxyl groups; more preferentially, the heterocyclic group is a dithiolane group optionally substituted with a (C₁-C₆)alkyl group optionally substituted with one or more hydroxyl, thiol or carboxyl groups.

Preferably, the reducing agent(s) are chosen from thioglycolic acid, thiolactic acid, cysteine, cysteamine, homocysteine, glutathione, thioglycerol, thiomalic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters and amides, notably glyceryl monothioglycolate, and mixtures thereof.

The thiol-based reducing agent(s) may notably be used in the form of salts, in particular salts of alkali metals such as the sodium and potassium salts, salts of alkaline-earth metals, for example the magnesium and calcium salts, the ammonium salts, the amine salts and the amino alcohol salts. Ammonium thioglycolate may thus be used as thiol-based reducing agent.

Particularly preferably, the thiol-based reducing agent(s) may be chosen from thioglycolic acid, thiolactic acid and cysteamine, and mixtures thereof.

More particularly preferably, the thiol-based reducing agent(s) may be chosen from thioglycolic acid, thiolactic acid, and mixtures thereof.

The composition applied to the keratin fibers during the step of permanent reshaping of the keratin fibers may comprise a total content of thiol-based reducing agents ranging from 1% to 30% by weight and preferably ranging from 5% to 25% by weight relative to the total weight of the composition.

According to a particular embodiment of the invention, the pH of the composition comprising the thiol-based reducing agent(s) is basic, preferably ranging from 8 to 10.

According to another particular embodiment of the invention, the pH of the composition comprising the thiol-based reducing agent(s) is acidic, preferably ranging from 1 to 6, more preferentially ranging from 2 to 5 and even more preferentially ranging from 2.5 to 4.

By way of example, the pH of the composition comprising the thiol-based reducing agent(s) may be equal to 3.5.

The composition comprising the thiol-based reducing agent(s) may advantageously be applied to the keratin fibers in an amount ranging from 0.1 g to 10 g of composition per gram of keratin fibers.

Non-Thiol Reducing Agents

The term “non-thiol reducing agent” means a reducing agent not bearing any thiol groups.

The non-thiol reducing agent(s) may preferably be chosen from sulfites, bisulfites, sulfinates, phosphines, sugars, reductones, hydrides and mixtures thereof.

The non-thiol reducing agent(s) may more preferentially be chosen from ammonium sulfites and bisulfites, and also from metal sulfites and bisulfites, even more preferentially from alkali metal or alkaline-earth metal sulfites and bisulfites, most preferentially from sodium sulfites and bisulfites.

Sulfinates that may be mentioned include sulfinic acid salts and benzenesulfinic acid salts, such as the sodium salts thereof. The sulfinic acid derivatives described in FR-A-2 814 948 may also be used. A preferred sulfinate compound is the disodium salt of 2-hydroxy-2-sulfinatoacetic acid.

Phosphines that may be mentioned include monophosphine and diphosphines, as described in FR-A-2 870 119. According to a particular embodiment of the present invention, the phosphine(s) may be chosen from the compounds of formula i-3 below:

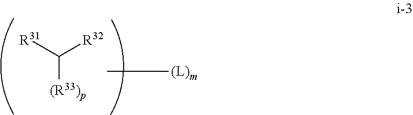

in which:
L is a linker (linking agent) which represents a covalent bond or a divalent hydrocarbon-based radical optionally comprising one or more heteroatoms chosen from an oxygen atom, a sulfur atom, a nitrogen atom and a silicon atom;
m is an integer equal to 0 or 1;
q is an integer equal to 1 or 2;
p is an integer equal to 0 or 1;
R³¹, R³² and R³³, which may be identical or different, represent:

a hydrogen atom;

a halogen atom;

a hydroxyl radical;

a carboxyl radical;

a hydrocarbon-based monovalent radical optionally comprising one or more heteroatoms chosen from a sulfur atom, an oxygen atom, a nitrogen atom, a phosphorus atom and a silicon atom, optionally substituted with one or more radicals chosen from:

a halogen atom;

a hydroxyl radical;

an alkoxy radical,

a haloalkyl radical,

an amino radical;

a carboxyl radical;

an alkoxycarbonyl radical;

an amido radical;

an alkylaminocarbonyl radical;

an acylamino radical;

a mono- or di(alkyl)amino radical;

a mono- or di(hydroxyalkyl)amino radical;

an N-aryl-N-alkylamino radical;

an aromatic or heteroaromatic ring, which is unsubstituted or substituted with one or more radicals chosen from a halogen atom, a hydroxyl radical, an alkoxy radical and a mono- or di(alkyl)amino radical;

a cyano radical;

a radical which increases the solubility of the phosphine in water, such as sulfonate, sulfinate, phosphonate or carboxylate radicals;

a substituted or unsubstituted, aromatic or nonaromatic heterocyclic radical;

a substituted or unsubstituted aryl radical;

a substituted or unsubstituted arylalkyl radical;

an arylalkyloxy radical;

a substituted or unsubstituted, aromatic or nonaromatic heterocyclic radical;

a silyl radical;

it being understood that:
when q=1, m=0 and p=1;
when q=2, m=1 and p=0 or 1, with:
when p=0, the linker L is attached to the phosphorus atom; and
when p=1, the linker L is attached to one of the radicals or R³¹, R³² or R³³, and acid-addition salts thereof.

In the compounds of formula i-3 as defined previously, when a radical is substituted, the substituents are chosen from a halo, a hydroxyl, an alkyl, a haloalkyl, an alkoxy, an amino, a mono- or dialkylamino, a mono- or dihydroxyalkylamino and a carboxyl. For example, the p-methoxyphenyl radical is a substituted aryl radical.

Preferably, the radicals R³¹, R³² and R³³ do not simultaneously represent a hydrogen atom.

Advantageously, but optionally, at least one of the radicals R³¹, R³² and R³³ denotes, as hydrocarbon-based radical, an optionally substituted alkyl radical.

According to a particular embodiment of the invention, R³¹, R³² and R³³ are chosen from a hydrogen atom; an alkyl radical; a cycloalkyl radical optionally substituted with one or more alkyl radicals; an alkoxy radical; an alkoxyalkyl radical; a haloalkyl radical; a cyanoalkyl radical; a hydroxyalkyl radical; a carboxyalkyl radical; a halogen atom; a hydroxyl radical; a carboxyl radical; an alkenyl radical; a mono- or dialkylamino radical; an N-aryl-N-alkylaminoalkyl radical; an aryl radical optionally substituted with one or more radicals chosen from an alkyl radical, an alkoxy radical, a mono- or dialkylamino radical, a mono- or dialkylaminoalkyl radical, a haloalkyl radical, a hydroxyl radical, a carboxyl radical, a halogen atom, and an aryl radical substituted with a mono- or dialkylaminoalkyl radical; an arylalkyl radical; an arylalkyloxy radical; a pyrrolidino radical; a furyl radical; a morpholino radical; a thienyl radical; a pyridyl radical; a trialkylsilyl radical; and an alkyl radical substituted with a pyrrolidino radical, a furyl radical, a morpholino radical or a thienyl radical.

By way of example, R³¹, R³² and R³³ may be chosen from a hydrogen atom, a methyl radical, an ethyl radical; a propyl radical; an isopropyl radical; an n-butyl radical; an isobutyl radical; a tert-butyl radical; an octyl radical; a cyclohexyl radical; a cyclopentyl radical; a methoxy radical; an ethoxy radical; a methoxypropyl radical; a chloroethyl radical; a cyanoethyl radical; a hydroxymethyl radical; a hydroxypropyl radical; a carboxyethyl radical; a chlorine atom; a hydroxyl radical; a carboxyl radical; a trifluoromethyl radical; a chloromethyl radical; an allyl radical; a vinyl radical; a dimethylamino radical; a diethylamino radical; a di(isopropyl)amino radical; a phenyl radical; an o-tolyl radical; an m-tolyl radical; a p-tolyl radical; a dimethylphenyl radical; a trimethylphenyl radical; an o-methoxyphenyl radical; an m-methoxyphenyl radical; a p-methoxyphenyl radical; a dimethoxyphenyl radical; a trimethoxyphenyl radical; an o (dimethylamino)phenyl radical; an m-(dimethylamino)phenyl radical; a p-(dimethylamino)phenyl radical; a di(tert-butyl)phenyl radical; a tri(tert-butyl)phenyl radical; a trifluoromethylphenyl radical; a bis(trifluoromethyl)phenyl radical; an o-fluorophenyl radical; an m-fluorophenyl radical; a p-fluorophenyl radical; an o-chlorophenyl radical; an m-chlorophenyl radical; a p-chlorophenyl radical; an o-hydroxyphenyl radical; an m-hydroxyphenyl radical; a p-hydroxyphenyl radical; a 4-(diethylaminomethyl)phenyl radical; a 3,5-dimethyl-4-methoxyphenyl radical; a 2-methylbiphenyl radical; a benzyl radical; a benzyloxy radical; a naphthyl radical; a morpholino radical; a morpholinomethyl radical; a pyrrolidino radical; a furyl radical; a pyridyl radical; a thienyl radical; a trimethylsilyl radical; a 2-(4-diethylaminomethylphenyl)phenyl radical; a 5-methyl-2-isopropylcyclohexyl radical; an N-methyl-N-phenylaminomethyl; and a carboxyphenyl radical.

The phosphines that may be used in the context of the invention may optionally be salified with strong mineral acids, for example HCl, HBr, H₂SO₄ or HBF₄, or organic acids, for example acetic acid, lactic acid, tartaric acid, citric acid or succinic acid.

According to a particular embodiment of the invention, the phosphine(s) that may be used in the context of the invention are chosen from monophosphines. For example, when the phosphine(s) are of formula i-3, q is then preferably equal to 1.

Examples of monophosphines that may be mentioned include tri(hydroxymethyl)-phosphine; tri(hydroxypropyl)phosphine; bis(hydroxymethyl)(phenyl)phosphine; allyldiphenylphosphine; benzyldiphenylphosphine; bis(3,4,5-trimethoxyphenyl)chlorophosphine; bis(3,4,5-trimethoxyphenyl)phosphine; benzyloxy(diisopropylamino)methylphosphine; bis(diisopropylamino)chlorophosphine; bis(2-cyanoethyl)phosphine; bis(3,5-di-tert-butylphenyl)chlorophosphine; bis(3,5-di-tert-butylphenyl)phosphine; bis(diethylamino)methylphosphine; bis(diethylamino)-chlorophosphine; bis(diethylamino)phenylphosphine; bis(3,5-dimethyl methoxyphenyl)chlorophosphine; bis(3,5-dimethyl-4-methoxyphenyl)phosphine; bis(3,5-dimethylphenyl)chlorophosphine; bis(3,5-dimethylphenyl)-diethylaminophosphine; bis(3,5-dimethylphenyl)phosphine; bis(3,5-ditrifluoromethylphenyl)chlorophosphine; bis(3,5-ditrifluoromethylphenyl)phosphine; bis(4-fluorophenyl)chlorophosphine; bis(2-furyl)chlorophosphine; bis(2-furyl)phosphine; bis(hydroxymethyl)phenylphosphine; bis(4-methoxyphenyl)phenylphosphine; bis(3,5-dimethylphenyl)phosphine; bis(3,5-di-tert-butylphenyl)chlorophosphine; bis(3,5-di-tert-butylphenyl)phosphine; bis(3,5-ditrifluoromethylphenyl)chlorophosphine; bis(3,5-ditrifluoromethylphenyl)phosphine; bis(4-fluorophenyl)chlorophosphine; bis(4-methoxyphenyl)chlorophosphine; bis(4-methoxyphenyl)phenylphosphine; bis(4-methylphenyl)chlorophosphine; bis(4-methylphenyl)phosphine; bis(4-trifluoromethylphenyl)chlorophosphine; bis(4-trifluoromethylphenyl)phosphine; bis(diethylamino)methylphosphine; bis(diethylamino)phenylphosphine; bis(hydroxymethyl)phenylphosphine; bis(o-tolyl)chlorophosphine; bis(o-tolyl)phosphine; bis(pyrrolidino)methylphosphine; butyldichlorophosphine; butyldiphenylphosphine; tert-butyldiphenylphosphine; cyclohexyl(diethylamino)chlorophosphine; cyclohexyl(dimethylamino)chlorophosphine; cyclohexyldichlorophosphine; cyclohexyldiphenylphosphine; 2-chloroethyldiphenylphosphine; 2-(dicyclohexylphosphino) biphenyl; 2-dicyclohexylphosphino-2′-(N, N-dimethylamino)biphenyl; diethylaminodiethylphosphine; dimethylaminodichlorophosphine; (4-dimethylaminophenyl)diphenylphosphine; N-[(diphenylphosphinyl)methyl]-N-methylaniline; o-diphenylphosphinobenzoic acid; 2-methoxy(dichlorophosphino)benzene; 4-methoxyphenyl(diethylamino)chlorophosphine; 4-methoxyphenyl(dimethylamino)chlorophosphine; (2-methoxyphenyl)methylphenylphosphine; 2-methoxyphosphinobenzene; (5-methyl-2-isopropylcyclohexyl)diphenylphosphine; triphenylphosphine; diallylphenylphosphine; dibenzylphosphine; dibutylphenylphosphine; dibutylphosphine; dicyclohexylchlorophosphine; dicyclohexylphenylphosphine; dicyclohexylphosphine; diethylchlorophosphine; diethylphenylphosphine; diethylphosphine; diisobutylphosphine; diisopropylchlorophosphine; diisopropylphosphine; dimethyl(phenyl)phosphine; dimethyl(trimethylsilyl)phosphine; dimethylchlorophosphine; diphenyl(o-tolyl)phosphine; diphenyl(p-tolyl)phosphine; diphenyl(trimethylsilyl)phosphine; diphenylchlorophosphine; diphenylphosphine; diphenylpropylphosphine; diphenylvinylphosphine; di-tert-butylchlorophosphine; di-tert-butylhydroxyphosphine; di-tert-butylmethylphosphine; di-tert-butylphenylphosphine; di-tert-butylphosphine; divinylphenylphosphine; ethyldichlorophosphine; ethyldiphenylphosphine; isopropyldichlorophosphine; methoxydiethoxyphosphine; methyldichlorophosphine; methyldiphenylphosphine; methylphenylchlorophosphine; phenylphosphine; propyldichlorophosphine; tert-butylbis(trimethylsilyl)phosphine; tert-butyldichlorophosphine; tert-butyldiethylphosphine; tert-butyldiphenylphosphine; tert-butylphosphine; tri(m-tolyl)phosphine; tri(o-tolyl)phosphine; tri(p-tolyl)phosphine; tricyclohexylphosphine; tricyclopentylphosphine; triethylphosphine; triisobutylphosphine; triisopropylphosphine; trimethylphosphine; tri-n-butylphosphine; tri-n-octylphosphine; tripropylphosphine; tris(1-naphthyl)phosphine; tris(2,4,6-trimethylphenyl)phosphine; tris(2,6-dimethoxyphenyl)phosphine; tris(2-carboxyethyl)phosphine; tris(2-cyanoethyl)phosphine; tris(2-furyl)phosphine; tris(2-methoxyphenyl)phosphine; tris(2-thienyl)phosphine; tris(3,5-dimethyl-4-methoxy)phosphine; tris(3-chlorophenyl)phosphine; tris(3-fluorophenyl)phosphine; tris(3-methoxyphenyl)phosphine; tris(3-methoxypropyl)phosphine; tris(4-chlorophenyl)phosphine; tris(4-fluorophenyl)phosphine; tris(4-methoxyphenyl)phosphine; tris(4-morpholino)phosphine; tris(hydroxymethyl)phosphine; tris(trimethylsilyl)phosphine; tris[3,5-bis(trifluoromethyl)phenyl]phosphine; tri-tert-butylphosphine; 2-cyanoethyldiphenylphosphine; 2-dicyclohexylphosphino-2′-methylbiphenyl; bis(2,4,6-trimethylphenyl)phosphine; and 2-(di-tert-butylphosphino)biphenyl.

Preferably, the monophosphines are chosen from trihydroxymethylphosphine; trihydroxypropylphosphine; and bis(hydroxymethyl)phenylphosphine.

According to another particular embodiment of the invention, the phosphine(s) that may be used in the context of the invention are diphosphines. When the phosphine(s) are of formula i-3, then q is preferably equal to 2.

Preferably, p is equal to 0 and the linker L is a covalent bond or a divalent radical that is chosen from a binaphthylene radical; a methylene radical; an ethylene radical; a propylene radical; a butylene radical; a pentylene radical; a hexylene radical; a phenylene radical; a meta-di methylenebenzene radical; an N-methyl-N′-methylhydrazo radical; a vinylene radical; and a diethyleneoxy radical.

As examples of diphosphines that may be used in the context of the invention, mention may be made of 2,2′-bis(dicyclohexylphosphino)-1,1′-binaphthyl; 2,2′-bis[bis(3,5-dimethylphenylphosphino)]-1,1′-binaphthyl; 1,4-bis[bis(3,5-dimethylphenyl)phosphino]butane; 1,2-bis[bis(3,5-dimethylphenyl)phosphino]ethane; bis[bis(3,5-dimethylphenyl)phosphino]methane; 1,5-bis[bis(3,5-dimethylphenyl)phosphino]pentane; 1,3-bis[bis(3,5-dimethylphenyl)phosphino]propane; 2,2′-bis[bis(3,5-ditrifluoromethylphenyl)phosphino]-1,1′-binaphthyl; 1,4-bis[bis(3,5-ditrifluoromethylphenyl)phosphino]butane; 1,2-bis[bis(3,5-ditrifluoromethylphenyl)phosphino]ethane; bis[bis(3,5-ditrifluoromethylphenyl)phosphino]methane; 1,5-bis-[bis(3,5-ditrifluoromethylphenyl)phosphino]pentane; 1,3-bis[bis(3,5-ditrifluoromethylphenyl)phosphino]propane; 1,2-bis(di-tert-butylphosphino)benzene; 1,4-bis(di-tert-butylphosphino)butane; 1,2-bis(di-tert-butylphosphino)ethane; 1,3-bis(di-tert-butylphosphinomethyl)benzene; 1,3-bis(di-tert-butylphosphino)propane; 1,2-bis(dichlorophosphino)benzene; 1,3-bis(dichlorophosphino)benzene; 1,4-bis(dichlorophosphino)benzene; 1,4-bis(dichlorophosphino)butane; 1,2-bis(dichlorophosphino)-1,2-dimethylhydrazine; 1,2-bis(dichlorophosphino)ethane; bis(dichlorophosphino)methane; 1,3-bis(dichlorophosphino)propane; 1,2-bis(dicyclohexylphosphino)benzene; 2,2′-bis(dicyclohexylphosphino)-1,1′-binaphthyl; 1,4-bis(dicyclohexylphosphino)butane; (2R,3R)bis(dicyclohexylphosphino)butane; (2S,3S)-bis(dicyclohexylphosphino)butane; 1,2-bis(dicyclohexylphosphino)ethane; bis(dicyclohexylphosphino)methane; 1,3-bis(dicyclohexylphosphino)propane; bis[2-(4-diethylaminomethylphenyl)phenylphosphino]ethyl ether; 1,2-bis(diethylphosphino)ethane; 1,2-bis(dimethylphosphino)benzene; 1,4-bis(dimethylphosphino)butane; 1,2-bis(dimethylphosphino)ethane; bis(dimethylphosphino)methane; 1,3-bis(dimethylphosphino)propane; 1,2-bis(diphenylphosphino)benzene; 1,3-bis(diphenylphosphino)benzene; 1,4-bis(diphenylphosphino)benzene; 2,2′-bis(diphenylphosphino)-1,1′-binaphthyl; 1,4-bis(diphenylphosphino)butane; 1,2-bis(diphenylphosphino)ethane; cis-1,2-bis(diphenylphosphino)ethylene; trans-1,2-bis(diphenylphosphino)ethylene; bis(2-diphenylphosphino)ethyl ether; 1,6-bis(diphenylphosphino)hexane; bis(diphenylphosphino)methane; 1,5-bis(diphenylphosphino)pentane; 1,3-bis(diphenylphosphino)propane; 1,2-bis(ditrifluoromethylphosphino)ethane; 1,2-bis[(2-methoxyphenyl)phenylphosphino]ethane; 1,2-bis(phenylphosphino)ethane; 1,3-bis(phenylphosphino) propane; bis-2-[(phenyl)(3-pyridyl)phosphinoethyl] ether; 1,2-bis(phosphino)benzene; 1,2-bis(phosphino)ethane; bis(phosphino)methane; 1,2-bis(trifluoromethyl)phosphino)ethane; bis(di-tert-butylphosphino)pentane; and tetraphenylbiphosphine.

According to a particular embodiment of the invention, the phosphine(s) that may be used in the context of the invention are soluble in a cosmetically acceptable medium.

Preferably, the phosphine(s) that may be used in the context of the invention are water-soluble.

In the context of the present invention, the term “water-soluble” refers to any phosphine whose solubility in water is greater than 0.01% by weight at 20° C. Preferably, the phosphine is trihydroxymethylphosphine.

Sugars that may be mentioned include ribose, glucose, maltose, galactose, lactose and xylose.

Reductones that may be mentioned include ascorbic acid and erythorbic acid.

Hydrides that may be mentioned include boron hydrides such as sodium borohydride, lithium hydride and phosphorous hydride. Hydride precursors, and in particular boron hydrides, such as diborane, tetraborane, pentaborane, decaborane and dodecaborane, may be used.

According to a particularly preferred embodiment, the non-thiol reducing agent(s) are chosen from sulfites, bisulfites, phosphines, and mixtures thereof.

Alkaline Agents

The step of permanent reshaping of keratin fibers may comprise the application to the keratin fibers of a composition comprising one or more alkaline agents chosen from mineral hydroxides, organic hydroxides, and mixtures thereof.

The mineral hydroxides are preferably chosen from alkali metal hydroxides, alkaline-earth metal hydroxides, transition metal hydroxides and mixtures thereof, more preferentially chosen from sodium hydroxide, lithium hydroxide, calcium hydroxide, magnesium hydroxide, barium hydroxide, strontium hydroxide, manganese hydroxide, zinc hydroxide and mixtures thereof.

Even more preferentially, the alkaline agent is sodium hydroxide.

Among the organic hydroxides, use will preferably be made of guanidinium hydroxide.

Guanidinium hydroxide is generally obtained by mixing, at the time of use, guanidine carbonate and calcium hydroxide.

According to a first variant, the composition according to the invention comprises guanidine carbonate and, at the time of use, this composition is then mixed with a composition comprising calcium hydroxide, known as an “activator”.

According to a second variant, the composition according to the present invention comprises calcium hydroxide and, at the time of use, this composition is then mixed with a composition comprising guanidine carbonate, known as an “activator”.

Preferably, the amount of alkaline agents chosen from mineral hydroxides, organic hydroxides and mixtures thereof is such that the composition has a pH ranging from 12 to 14.

The composition may comprise a total content of alkaline agents chosen from mineral hydroxides, organic hydroxides and mixtures thereof ranging from 1% to 10% by weight relative to the total weight of the composition.

The composition comprising the alkaline agent(s) may advantageously be applied to the keratin fibers in an amount ranging from 0.1 g to 10 g of composition per gram of keratin fibers.

Substep of rinsing and/or drying the keratin fibers Step ii) of permanent reshaping of keratin fibers may comprise a substep of rinsing and/or drying the keratin fibers, preferably a substep of rinsing and then a substep of drying.

The substep of rinsing may be a substep of rinsing with water or using an aqueous composition comprising a chemical oxidizing agent. Hydrogen peroxide may be used as chemical oxidizing agent.

The chemical oxidizing agent may be applied to the keratin fibers in the form of an aqueous composition comprising from 0.3% to 12% by weight (1 to 40 volumes), preferably from 0.6% to 2% by weight (2 to 7 volumes) of hydrogen peroxide.

The drying substep may be performed using a hairdryer, a styling hood or by drying naturally. The drying substep may advantageously be performed at a temperature ranging from 20° C. to 70° C. The drying substep may be performed using a hairdryer and a brush (blow-drying).

Heat-Treatment Substep

Step ii) of permanent reshaping of the keratin fibers may comprise a substep of heat treatment of the keratin fibers using a heating tool.

The heating tool is preferably maintained at a temperature ranging from 65° C. to 250° C., more preferentially from 80° C. to 230° C., even more preferentially from 150° C. to 230° C., most preferentially from 160° C. to 230° C., most preferentially from 180° C. to 230° C. The heating tool is preferably chosen from a straightening iron (or flat tongs), a steam iron, a styling hood, a hairdryer, an infrared ray dispenser and a heating comb, more preferentially a steam iron or a straightening iron, even more preferentially a straightening iron.

The term “iron” means a device for heating keratin fibers by placing said fibers in contact with the heating device. The end of the iron which comes into contact with the keratin fibers generally has two flat surfaces. These two surfaces may be made of metal or of ceramic. In particular, these two surfaces may be smooth or crimped or curved. As examples of irons that may be used in the process according to the present invention, mention may be made of any type of flat iron, and in particular, in a nonlimiting manner, those described in patents U.S. Pat. Nos. 5,957,140 and 5,046,516.

The term “steam iron” means an iron which comprises a device which emits steam and which applies this steam before, during or after the straightening operation.

An example of steam irons that may be mentioned is irons of the Steampod type from Rowenta.

Advantageously, steam is applied to the keratin fibers at a flow rate of less than 5 g/min, in particular of between 1 and 4 g/min.

The straightening or steam iron may be applied by successive separate strokes lasting a few seconds or by gradual movement or sliding along the locks.

The straightening or steam iron may be applied in a continuous movement from the root to the end of the hair, in one or more strokes, in particular in two to twenty strokes. The duration of each stroke of the straightening or steam iron may range from 2 seconds to 1 minute.

The straightening or steam iron is preferably applied on keratin fibers that have been dried beforehand.

According to a preferred embodiment, step ii) of permanent reshaping of the keratin fibers comprises a rinsing substep and then a drying substep and then a substep of heat treatment using a straightening iron.

Additional Features Regarding the Process

Composition (A) may be applied to wet or dry keratin fibers.

Bath Ratio

Composition (A) may advantageously be applied to the keratin fibers in an amount ranging from 0.1 g to 10 g of composition (A) per gram of keratin fibers.

Preferably, composition (A) may be applied to the keratin fibers in an amount ranging from 0.2 g to 5 g of composition (A) per gram of keratin fibers.

Leave-on Time Step i′)

The process preferably also comprises, between steps i) and ii), a step i′) consisting in leaving composition (A) to stand on the keratin fibers for a time ranging from 1 min to 60 min, more preferentially ranging from 3 min to 40 min and even more preferentially ranging from 3 min to 20 min.

The leave-on time step may take place at a temperature ranging from 15° C. to 45° C., preferably at room temperature (25° C.). The leave-on time may take place under an occlusive system. A nonlimiting example of an occlusive system that may be mentioned is an occlusive system of envelope type made of aluminum or plastic film or a hair cap with or without holes.

Rinsing and/or Drying Step i″)

The process may also comprise, after step i) or i′) and before step ii), a step i″) of rinsing and/or drying the keratin fibers, preferably a step i″) of drying the keratin fibers.

According to a preferred embodiment, the process does not comprise a rinsing step i″) between step i) or i′) and step ii).

The term “rinsing step” means a step of rinsing with water.

The drying step may be performed using absorbent paper, a hairdryer or a styling hood or by drying naturally.

Step of Dyeing or Bleaching the Keratin Fibers

The process according to the present invention may also comprise:
after step i) or i′) or i″) and before step ii); and/or
after step ii)

a step of dyeing or bleaching the keratin fibers.

Preferably, the process according to the present invention comprises, after step i) or i′) or i″) and before step ii), a step of dyeing or bleaching the keratin fibers.

The dyeing or bleaching step may comprise the application to the keratin fibers of a dyeing or bleaching composition.

Chemical Oxidizing Agent

The dyeing or bleaching composition may comprise at least one chemical oxidizing agent.

Preferably, the chemical oxidizing agent is chosen from hydrogen peroxide, urea peroxide, alkali metal bromates, peroxygenated salts, peracids and precursors thereof, and mixtures thereof.

More preferentially, the chemical oxidizing agent is chosen from hydrogen peroxide, peroxygenated salts, and mixtures thereof.

Even more preferentially, the chemical oxidizing agent is chosen from hydrogen peroxide, persulfates, perborates or percarbonates of alkali metals or alkaline-earth metals or of ammonium, and mixtures thereof.

More preferentially, the chemical oxidizing agent is hydrogen peroxide.

Examples of peroxygenated salts that may be mentioned include sodium, potassium or ammonium persulfates and mixtures thereof.

When the composition is a bleaching composition, it may preferably comprise hydrogen peroxide and a peroxygenated salt.

The dyeing or bleaching composition may comprise a total content of chemical oxidizing agents ranging from 0.5% to 60% by weight, preferably ranging from 0.5% to 40% by weight, more preferentially ranging from 1% to 30% by weight, relative to the total weight of the dyeing or bleaching composition.

Liquid Fatty Substances

The dyeing or bleaching composition may also comprise one or more fatty substances that are liquid at room temperature (25° C.) and at atmospheric pressure (1.013×10⁵ Pa), other than salified fatty acids.

The term “fatty substance” means an organic compound that is insoluble in water at room temperature (25° C.) and at atmospheric pressure (1.013×10⁵ Pa) (solubility of less than 5% by weight, preferably less than 1% by weight and even more preferentially less than 0.1% by weight). They bear in their structure at least one hydrocarbon-based chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The term “oil” means a “fatty substance” that is liquid at room temperature (25° C.) and at atmospheric pressure (1.013×10⁵ Pa).

The term “nonsilicone fatty substance” refers to a fatty substance not containing any Si—O bonds and the term “silicone fatty substance” refers to a fatty substance containing at least one Si—O bond.

The liquid fatty substances that may be used in the dyeing or bleaching composition are different from salified fatty acids, i.e. they can be present in the composition in the form of free fatty acids. In other words, these fatty substances do not contain any salified carboxylic acid groups (—C(O)O—). In particular, these fatty substances are neither polyoxyethylenated nor polyglycerolated.

Preferably, the fatty substances are different from non-salified fatty acids.

More particularly, the liquid fatty substances according to the invention are chosen from C₆ to C₁₆ liquid hydrocarbons, liquid hydrocarbons comprising more than 16 carbon atoms, nonsilicone oils of animal origin, oils of triglyceride type of plant or synthetic origin, fluoro oils, liquid fatty alcohols, liquid fatty acid and/or fatty alcohol esters other than triglycerides, and silicone oils, and mixtures thereof.

It is recalled that the fatty alcohols and esters more particularly contain at least one saturated or unsaturated, linear or branched hydrocarbon-based group, comprising 6 to 30 and better still from 8 to 30 carbon atoms, which is optionally substituted, in particular, with one or more hydroxyl groups (in particular 1 to 4). If they are unsaturated, these compounds may comprise one to three conjugated or unconjugated carbon-carbon double bonds.

As regards the C₆ to C₁₆ liquid hydrocarbons, they are linear, branched or optionally cyclic, and are preferably alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly, polydecenes, hydrogenated polyisobutene such as Parleam®, and mixtures thereof.

A hydrocarbon-based oil of animal origin that may be mentioned is perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, caprylic/capric acid triglycerides, for instance those sold by the company Stéarinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The liquid fatty alcohols may more particularly be chosen from linear or branched, saturated or unsaturated alcohols, preferably unsaturated or branched alcohols, including from 6 to 30 carbon atoms and preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or of fatty alcohols other than the triglycerides mentioned above, mention may be made notably of esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6 and more advantageously greater than or equal to 10.

Preferably, for the esters of monoalcohols, at least one from among the alcohol and the acid from which the esters of the invention are derived is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl palmitate, isopropyl palmitate, 2-ethylhexyl palmitate, 2-octyldecyl palmitate, alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of ethyl palmitate, isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Still within the context of this variant, use may also be made of esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols. Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The dyeing or bleaching composition may also comprise, as fatty ester, sugar esters and diesters of C₆ to C₃₀ and preferably C₁₂ to C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmito-stearate esters More particularly, use is made of monoesters and diesters and notably sucrose, glucose or methylglucose mono- or di-oleates, stearates, behenates, oleopalmitates, linoleates, linolenates and oleostearates, and mixtures thereof.

An example that may be mentioned is the product sold under the name Glucate® DO by the company Amerchol, which is a methylglucose dioleate.

Preferably, use will be made of a liquid ester of a monoacid and of a monoalcohol.

The silicone oils that may be used in the dyeing or bleaching composition may be volatile or nonvolatile, cyclic, linear or branched silicone oils, which are unmodified or modified with organic groups, and preferably have a viscosity from 5×10⁻⁶ to 2.5 m²/s at 25° C., and preferably 1×10⁻⁶ to 1 m²/s.

Preferably, the silicone oils are chosen from polydialkylsiloxanes, notably polydimethylsiloxanes (PDMS), and liquid polyorganosiloxanes including at least one aryl group.

These silicone oils may also be organomodified. The organomodified silicone oil(s) that may be used in the dyeing or bleaching composition are preferably liquid silicones as defined previously and including in their structure one or more organofunctional groups attached via a hydrocarbon-based group, chosen, for example, from amine groups and alkoxy groups.

Organopolysiloxanes are defined in greater detail in Walter Noll's Chemistry and Technology of Silicones (1968), Academic Press. They may be volatile or nonvolatile. When they are volatile, the silicone oils are more particularly chosen from those with a boiling point of between 60° C. and 260° C., and even more particularly from:

(i) cyclic polydialkylsiloxanes including from 3 to 7 and preferably from 4 to 5 silicon atoms. These are, for example, octamethylcyclotetrasiloxane notably sold under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Volatile Silicone® FZ 3109 sold by the company Union Carbide.

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane notably sold under the name SH 200 by the company Toray Silicone. Silicones falling within this category are also described in the article published in Cosmetics and Toiletries, Vol. 91, January 76, pages 27-32—Todd & Byers Volatile Silicone Fluids for Cosmetics.

Nonvolatile polydialkylsiloxanes are preferably used.

These silicone oils are more particularly chosen from polydialkylsiloxanes, among which mention may be made mainly of polydimethylsiloxanes bearing trimethylsilyl end groups. The viscosity of the silicones is measured at 25° C. according to ASTM standard 445 Appendix C.

Among these polydialkylsiloxanes, mention may be made, in a nonlimiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

The organomodified silicones that may be used in the dyeing or bleaching composition are silicones as defined previously and including in their structure one or more organofunctional groups attached via a hydrocarbon-based group.

As regards the liquid polyorganosiloxanes including at least one aryl group, they may notably be polydiphenylsiloxanes, and polyalkylarylsiloxanes functionalized with the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may be made of polyorganosiloxanes including:
substituted or unsubstituted amine groups, such as the products sold under the names GP 4 Silicone Fluid and GP 7100 by the company Genesee or the products sold under the names Q2 8220 and Dow Corning 929 or 939 by the company Dow Corning. The substituted amine groups are, in particular, C₁ to C₄ aminoalkyl groups;
alkoxy groups;
hydroxyl groups.

The liquid fatty substance(s) are preferentially chosen from liquid hydrocarbons containing more than 16 carbon atoms, plant oils, liquid fatty alcohols and liquid fatty esters, silicone oils and mixtures thereof.

Preferentially, the liquid fatty substance(s) are chosen from liquid hydrocarbons comprising more than 16 carbon atoms, in particular liquid petroleum jelly.

In a particular embodiment, the total content of liquid fatty substance(s) included in the dyeing or bleaching composition is greater than or equal to 20% by weight, preferably greater than or equal to 30% by weight, more preferentially greater than or equal to 35% by weight, relative to the total weight of the dyeing or bleaching composition.

More preferentially, the total content of liquid fatty substance(s) included in the dyeing or bleaching composition ranges from 20% to 80% by weight, and preferably from 30% to 70% by weight, relative to the total weight of the dyeing or bleaching composition.

Alkaline Agents

The dyeing or bleaching composition may optionally also comprise one or more alkaline agents.

Preferably, the dyeing or bleaching composition comprises one or more organic or mineral alkaline agents.

The mineral alkaline agent(s) are preferably chosen from aqueous ammonia, alkali metal carbonates or bicarbonates such as sodium or potassium carbonates and sodium or potassium bicarbonates, sodium or potassium hydroxide, alkali metal silicates or metasilicates such as sodium or potassium silicates or metasilicates, or mixtures thereof.

The organic alkaline agent(s) are preferably chosen from organic amines with a pK_{b} at 25° C. of less than 12, preferably less than 10 and even more advantageously less than 6. It should be noted that it is the pK_{b} corresponding to the function which has the highest basicity. In addition, the organic amines do not comprise any alkyl or alkenyl fatty chains comprising more than ten carbon atoms.

The organic alkaline agent(s) are chosen, for example, from alkanolamines, oxyethylenated and/or oxypropylenated ethylenediamines, amino acids and the compounds of formula (III) below:

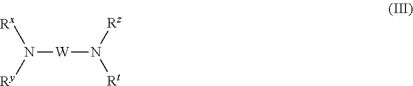

in which formula (III):
W is a divalent C₁-C₆ alkylene group optionally substituted with a hydroxyl group or a (C₁-C₆)alkyl group, and/or optionally interrupted with one or more heteroatoms such as oxygen or NRu;
R^{x}, R^{y}, R^{z} R^{t} and R^{u}, which may be identical or different, represent a hydrogen atom or a group chosen from (C₁-C₆)alkyl, C₁-C₆ hydroxyalkyl or C₁-C₆ aminoalkyl.

Examples of amines of formula (III) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁ to C₈ alkyl groups bearing one or more hydroxyl radicals.

Organic amines chosen from alkanolamines such as monoalkanolamines, dialkanolamines or trialkanolamines comprising one to three identical or different C₁ to C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form.

As amino acids that may be used in the dyeing or bleaching composition, mention may notably be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (IV) below, and also salts thereof:

R—CH₂—CH(NH₂)—C(O)—OH (IV)

in which R represents a group chosen from imidazolyl, preferably imidazolyl-4-yl; aminopropyl; aminoethyl; —(CH₂)₂N(H)—C(O)—NH₂; and —(CH₂)₂—N(H)—C(NH)—NH₂.

The compounds corresponding to formula (IV) are histidine, lysine, arginine, ornithine and citrulline.

The organic amine may also be chosen from organic amines of heterocyclic type.

Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that may be used in the present invention, mention may notably be made of carnosine, anserine and balenine.

The organic amine may also be chosen from compounds including a guanidine function. As amines of this type that may be used in the present invention, besides arginine, which has already been mentioned as an amino acid, mention may be made notably of creatine, creatinine, 1,1-dimethylguanidine, 1,1-diethylguanidine, glycocyamine, metformin, agmatine, n-amidinoalanine, 3-guanidinopropionic acid, 4-guanidinobutyric acid and 2-([amino(imino)methyl]amino)ethane-1-sulfonic acid.

Hybrid compounds that may be mentioned include the salts of the amines mentioned previously with acids such as carbonic acid or hydrochloric acid.

Guanidine carbonate or monoethanolamine hydrochloride may be used in particular.

Preferably, the alkaline agent(s) present in the dyeing or bleaching composition are chosen from aqueous ammonia, alkanolamines, alkali metal silicates, alkali metal metasilicates and mixtures thereof.

More preferentially, the alkaline agent present in the dyeing composition is monoethanolamine.

More preferentially, the alkaline agent present in the bleaching composition is chosen from sodium silicate, sodium metasilicate and mixtures thereof.

The total content of alkaline agents included in the dyeing or bleaching composition may range from 0.01% to 30% by weight, and preferably from 0.1% to 20% by weight, relative to the total weight of the dyeing or bleaching composition.

Solvents

The dyeing or bleaching composition may optionally also comprise one or more organic solvents.

Examples of organic solvents that may be mentioned include linear or branched C₂ to C₄ alkanols, such as ethanol and isopropanol; glycerol; polyols and polyol ethers, for instance 2-butoxyethanol, propylene glycol, hexylene glycol, dipropylene glycol, propylene glycol monomethyl ether, diethylene glycol monomethyl ether and monoethyl ether, and also aromatic alcohols or ethers, such as benzyl alcohol or phenoxyethanol, and mixtures thereof.

The organic solvent(s) may be present in the dyeing or bleaching composition in a content ranging from 0.01% to 30% by weight, preferably ranging from 2% to 25% by weight, relative to the total weight of the dyeing or bleaching composition.

Step of Dyeing the Keratin Fibers

The dyeing step may comprise the application to the keratin fibers of a dye composition.

The dye composition may comprise at least one coloring agent chosen from oxidation dyes, direct dyes, and mixtures thereof, preferably from oxidation dyes.

Oxidation Dyes

The oxidation dyes are generally chosen from one or more oxidation bases optionally combined with one or more coupling agents (also known as couplers).

Oxidation Bases

The dye composition may optionally comprise one or more oxidation bases advantageously chosen from those conventionally used in the dyeing of keratin fibers.

By way of example, the oxidation bases are chosen from para-phenylenediamines, bis(phenyl)alkylenediamines, para-aminophenols ortho-aminophenols and heterocyclic bases, and the corresponding addition salts.

Among the para-phenylenediamines that may be mentioned are, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N, N-dimethyl-para-phenylenediamine, N, N-diethyl-para-phenylenediamine, N, N-dipropyl-para-phenylenediamine, 4-amino-N,N-diethyl-3-methylaniline, N,N-bis(β-hydroxyethyl)-para-phenylenediamine, 4-N, N-bis(β-hydroxyethyl)amino-2-methylaniline, 4-N, N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N, N-dimethyl-3-methyl-para-phenylenediamine, N-ethyl-N-(β-hydroxyethyl)-para-phenylenediamine, N-β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the corresponding addition salts with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the corresponding addition salts with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N, N′-bis(β-hydroxyethyl)-N, N′-bis(4′-aminophenyl)ethylenediamine, N, N′-bis(4-aminophenyl)tetramethylenediamine, N, N′-bis(β-hydroxyethyl)-N, N′-bis(4-aminophenyl)tetramethylenediamine, N, N′-bis(4-methylaminophenyl)tetramethylenediamine, N, N′-bis(ethyl)-N, N′-bis(4′-amino-3′-methylphenyl)ethylenediamine and 1,8-bis(2,5-diaminophenoxy)-3,6-dioxaoctane, and the corresponding addition salts.

Among the para-aminophenols that are mentioned are, for example, para-aminophenol, 4-amino-3-methylphenol, 4-amino-3-fluorophenol, 4-amino-3-chlorophenol, 4-amino-3-hydroxymethyl phenol, 4-amino-2-methylphenol, 4-amino-2-hydroxymethyl phenol, 4-amino-2-methoxymethyl phenol, 4-amino-2-aminomethylphenol, 4-amino-2-(8-hydroxyethylaminomethyl)phenol and 4-amino-2-fluorophenol, and the corresponding addition salts with an acid.

Among the ortho-aminophenols that may be mentioned, for example, are 2-aminophenol, 2-amino-5-methylphenol, 2-amino-6-methylphenol and 5-acetamido-2-aminophenol, and the corresponding addition salts.

Among the heterocyclic bases that may be mentioned, for example, are pyridine, pyrimidine and pyrazole derivatives.

Among the pyridine derivatives that may be mentioned are the compounds described, for example, in patents GB 1 026 978 and GB 1 153 196, for example 2,5-diaminopyridine, 2-(4-methoxyphenyl)amino-3-aminopyridine and 3,4-diaminopyridine, and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases that are useful in the present invention are chosen from 3-aminopyrazolo[1,5-a]pyridines and are preferably substituted on carbon atom 2 with:

a) a (di)(C₁-C₆)(alkyl)amino group, said alkyl group possibly being substituted with at least one hydroxyl, amino or imidazolium group;

b) an optionally cationic 5- to 7-membered heterocycloalkyl group comprising from 1 to 3 heteroatoms, optionally substituted with one or more (C₁-C₆)alkyl groups such as a di(C₁-C₄)alkylpiperazinium group; or

c) a (C₁-C₆)alkoxy group optionally substituted with one or more hydroxyl groups, such as a β-hydroxyalkoxy group, and the corresponding addition salts.

Among the pyrimidine derivatives that may be mentioned are the compounds described, for example, in patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FRA-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the corresponding addition salts. Use may also be made of 4,5-diamino-1-(β-methoxyethyl)pyrazole.

A 4,5-diaminopyrazole will preferably be used and even more preferentially 4,5-diamino-1-(β-hydroxyethyl)pyrazole and/or a corresponding salt.

The pyrazole derivatives that may also be mentioned comprise diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol one and/or a corresponding salt.

Heterocyclic bases that will preferably be used are 4,5-diamino-1-(β-hydroxyethyl)pyrazole and/or 2,3-diamino-6,7-dihydro-1H,5H-pyrazolo[1,2-a]pyrazol one and/or a corresponding salt.

Coupling Agents

The dye composition may optionally comprise one or more coupling agents advantageously chosen from those conventionally used in the dyeing of keratin fibers.

Among these coupling agents, mention may be made in particular of meta-phenylenediamines, meta-aminophenols, meta-diphenols, naphthalene-based coupling agents and heterocyclic coupling agents, and also the corresponding addition salts.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylami no)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1-H-3-methyl pyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts with an acid and the corresponding mixtures.

In general, the addition salts of oxidation bases and coupling agents that may be used in the context of the invention are chosen in particular from the addition salts with an acid such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of the dye composition, and preferably from 0.005% to 5% by weight relative to the total weight of the dye composition.

The coupling agent(s), if they are present, each advantageously represent from 0.001% to 10% by weight relative to the total weight of the dye composition, and preferably from 0.005% to 5% by weight relative to the total weight of the dye composition.

Direct Dyes

The dye composition may optionally comprise one or more direct dyes.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (IIIa) and (III′a), the azo cationic dyes (IVa) and (IV′a) and the diazo cationic dyes (Va) below:

| | | |
|---|---|---|
| Het⁺—C(R^{a})═N—N(R^{b})—Ar, | Het⁺—N(R^{a})—N═C(R^{b})—Ar, | Het⁺—N═N—Ar, An⁻ |
| An⁻ | An⁻ (III′a) | (IVa) |
| (IIIa) | | |
| Ar⁺—N═N—Ar″, An⁻ | and | Het⁺—N═N—Ar′—N═N—Ar, |
| (IV′a) | | An⁻ |
| | | (Va) |

in which formulae (IIIa), (III′a), (IVa), (IV′a) and (Va):
Het⁺ represents a cationic heteroaryl radical, preferably bearing an endocyclic cationic charge, such as imidazolium, indolium or pyridinium, which is optionally substituted, preferably with one or more (C₁-C₈)alkyl groups such as methyl;
Ar⁺ represents an aryl radical, such as phenyl or naphthyl, bearing an exocyclic cationic charge, preferably ammonium, particularly tri(C₁-C₈)alkylammonium such as trimethylammonium;
Ar represents an aryl group, in particular phenyl, which is optionally substituted, preferably with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or, as a variant, Ar represents a julolidine group;
Ar′ represents an optionally substituted divalent (hetero)arylene group such as phenylene, particularly para-phenylene, or naphthalene, which are optionally substituted, preferably with one or more (C₁-C₈)alkyl, hydroxyl or (C₁-C₈)alkoxy groups;
Ar″ represents an optionally substituted (hetero)aryl group such as phenyl or pyrazolyl, which are optionally substituted, preferably with one or more (C₁-C₈)alkyl, hydroxyl, (di)(C₁-C₈)(alkyl)amino, (C₁-C₈)alkoxy or phenyl groups;
R^{a} and R^{b}, which may be identical or different, represent a hydrogen atom or a (C₁-C₈)alkyl group, which is optionally substituted, preferably with a hydroxyl group; or, as a variant, the substituent R^{a} with a substituent of Het⁺ and/or R^{b} with a substituent of Ar and/or R^{a} with R^{b} form, together with the atoms that bear them, a (hetero)cycloalkyl; in particular, R^{a} and R^{b} represent a hydrogen atom or a (C₁-C₄)alkyl group, which is optionally substituted with a hydroxyl group;
An⁻ represents an anionic counterion, such as mesylate or halide.

Mention may be made in particular of the azo and hydrazono cationic dyes bearing an endocyclic cationic charge of formulae (IIIa), (III′a) and (IVa) as defined previously. More particularly those of formulae (IIIa), (III′a) and (IVa) derived from the dyes described in patent applications WO 95/15144, WO 95/01772 and EP-714954.

Preferably, the cationic part is derived from the following derivatives:

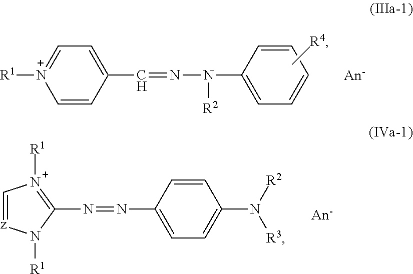

formulae (IIIa-1) and (IVa-1) with:
R¹ representing a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as an optionally substituted (C₁-C₈)alkyl, optionally substituted (C₁-C₈)alkoxy, or (di)(C₁-C₈)(alkyl)amino group optionally substituted on the alkyl group(s) with a hydroxyl group; in particular, R⁴ represents a hydrogen atom;
Z represents a CH group or a nitrogen atom, preferentially CH;
An⁻ represents an anionic counterion, such as mesylate or halide.

In particular, the dye of formulae (IIIa-1) and (IVa-1) is chosen from Basic Red 51, Basic Yellow 87 and Basic Orange 31 or corresponding derivatives:

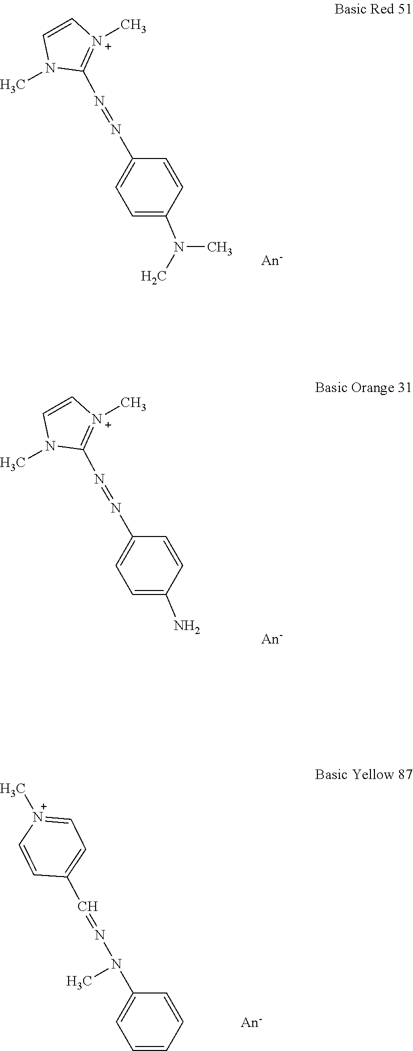

Among the natural direct dyes that may be used according to the invention, mention may be made of hennotannic acid, juglone, alizarin, purpurin, carminic acid, kermesic acid, purpurogallin, protocatechaldehyde, indigo, isatin, curcumin, spinulosin, apigenidin and orcein. Extracts or decoctions containing these natural dyes and in particular henna-based poultices or extracts may also be used.

When they are present, the direct dye(s) more particularly represent from 0.001% to 10% by weight and preferably from 0.005% to 5% by weight relative to the total weight of the dye composition.

The dyeing or bleaching composition may also optionally comprise one or more additives, different from the compounds described previously, among which mention may be made of cationic, anionic, nonionic or amphoteric polymers or mixtures thereof, antidandruff agents, antiseborrhea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, notably polymeric thickeners, opacifiers or nacreous agents, antioxidants, hydroxy acids, fragrances, preserving agents, pigments and ceramides.

Composition (A)

According to a second aspect, a subject of the present invention is composition (A) as defined previously.

Uses

According to a third aspect, a subject of the present invention is the use of composition (A) as defined previously, as a pretreatment composition of a process for permanently reshaping keratin fibers, preferably as a pretreatment composition of a process for straightening or relaxing keratin fibers.

The keratin fibers may be sensitized keratin fibers, notably keratin fibers which have undergone a dyeing or bleaching treatment beforehand.

According to a fourth aspect, a subject of the present invention is the use of a composition (A) as defined previously, for protecting keratin fibers, preferably for protecting them against breakage, during a treatment for permanently reshaping the keratin fibers.

Multi-Compartment Device (Kit)

According to a fifth aspect, a subject of the present invention is a multi-compartment device (kit) comprising:
a first compartment containing a composition (A) as defined previously; and
a second compartment containing a composition comprising at least one thiol-based or non-thiol reducing agent or at least one alkaline agent chosen from mineral hydroxides, organic hydroxides and mixtures thereof; and
optionally a third compartment containing a composition comprising at least one chemical oxidizing agent; and
optionally a fourth compartment containing a composition comprising at least one coloring agent chosen from oxidation dyes, direct dyes and mixtures thereof.

The optional technical features described previously concerning the thiol-based or non-thiol reducing agent, the alkaline agent, the chemical oxidizing agent and the coloring agent also apply to the compositions included in the second, third and fourth compartment.

### EXAMPLES

The examples that follow allow the invention to be understood more clearly, without, however, being limiting in nature. In the examples that follow, unless otherwise indicated, all the amounts are shown as mass percentages relative to the total weight of the composition.

### Example 1

The following pretreatment composition was prepared:

TABLE 1

| Ingredients | A1 |
|---|---|
| Glycine | 10 |
| Sodium hydroxide | qs pH = 9.2 |
| Water | qs 100 |

Application Protocol

Two 2.7 g locks of type IV Brazilian hair were used: lock 1 (Control) and lock 2 (Invention). Locks 1 and 2 underwent a bleaching treatment followed by a straightening treatment and then another bleaching treatment. Lock 2 (Invention) also underwent a pretreatment using composition A1 before the bleaching and straightening treatments.

1. Each of the locks was combed with a comb according to the following routine: 10 strokes at the coarse-toothed end and then 10 strokes at the fine-toothed end so as to remove the unattached hair strands.

2. The locks were then placed on a hotplate thermostatically regulated at 33° C.

3. The pretreatment composition A1 was applied to lock 2 with a bath ratio of 2 g of composition per 1 g of hair.

4. After a leave-on time of 5 minutes, lock 2 was blotted dry using a Kimtech 7505 absorbent paper towel.

5. A bleaching composition was prepared by mixing 1 part by weight of a bleaching powder based on persulfates with 2 parts by weight of aqueous hydrogen peroxide solution (30 volumes) and was then applied to lock 1 and to lock 2. The bath ratio is 10 g of composition per 1 g of hair.

6. Each of the locks was then wrapped in aluminum foil and then returned onto the hotplate at 33° C.

7. After a leave-on time of 50 minutes, the locks were rinsed with water and washed with L′Oreal Blond Studio shampoo and then blotted dry with a Kimtech 7505 absorbent paper towel.

8. A thiolactic acid solution at 8% by weight at pH 3.5 was then applied to lock 1 and to lock 2 laid flat on aluminum foil at room temperature. The bath ratio is 2 g of composition per 1 g of hair.

9. After a leave-on time of 30 minutes, the locks were rinsed with water and then dried with a hairdryer.

10. Once dry, a straightening iron maintained at a temperature of 230° C. was passed 10 times through each of the locks.

11. The locks were then treated with 4.5% aqueous hydrogen peroxide solution at acidic pH (bath ratio of 2 g of composition per 1 g of hair) to 10 minutes and were then rinsed and washed with Gamier Ultra Doux shampoo.

12. Steps 8 to 11 were repeated once.

13. Steps 5 to 7 were repeated once.

Evaluation of the Breakage of the Hair

The percentage of breakage of the treated hair was determined by means of a blow-drying breakage test. To do this, the following treatment was performed three times in succession on each of the locks:

1. wet the lock with water;

2. dry the lock by performing blow-drying;

3. wash the lock with 0.4 g of Gamier Ultra Doux shampoo per gram of hair.

The locks were weighed before and after the blow-drying breakage test after drying at 60° C. in an oven for 20 minutes.

The percentage of breakage of each of the locks is expressed by means of the following equation:

$$\% breakage = 100 - \frac{{m}_{1}}{{m}_{0}} * 100$$

in which:
m₀ is the mass of the lock measured before the blow-drying breakage test; and
m₁ is the mass of the lock measured after the blow-drying breakage test.

TABLE 2

| Type of lock | m₀ (mg) | m₁ (mg) | % breakage |
|---|---|---|---|
| Lock 1 | 3672 | 2843 | 22.58 |
| (Control) | | | |
| Lock 2 | 3684 | 3502 | 4.94 |
| (Invention) | | | |

The results show a marked decrease in breakage on blow-drying of the hair for the lock treated by means of the process according to the present invention comprising a pretreatment step using a composition comprising glycine at high concentration, relative to the same process without the pretreatment step.

1. A process for treating keratin fibers, comprising the following successive steps:
i) a step of applying to the keratin fibers a composition (A) comprising one or more amino acids chosen from the compounds of formula (I₁), salts thereof and mixtures thereof: 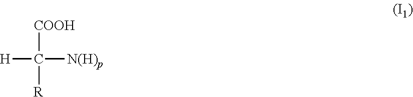
in which formula (I₁)
p is an integer equal to 1 or 2;
when p=1, R forms with the nitrogen atom a saturated 5- to 8-membered, preferably 5-membered, heterocycle, this ring possibly being optionally substituted with at least one group chosen from hydroxyl or (C₁-C₄)alkyl;
when p=2, R represents:
a hydrogen atom; or
a (C₁-C₁₂)alkyl group, preferably a (C₁-C₄)alkyl group, interrupted with at least one heteroatom or group chosen from —S—, —NH— or —C(NH)— and/or substituted with at least one group chosen from hydroxyl, amino or —NH—C(NH)—NH₂;
the amino acid(s) being present in composition (A) in a total content of at least 5% by weight relative to the total weight of composition (A);
ii) a step of permanent reshaping of the keratin fibers, preferably a step of straightening or relaxing the keratin fibers.

2. The process as claimed in claim 1, in which the amino acid(s) are chosen from glycine, proline, methionine, serine, arginine, lysine, salts thereof and mixtures thereof, preferably from glycine, proline, methionine, serine, salts thereof and mixtures thereof; more preferentially, the amino acid is glycine.

3. The process as claimed in either of the preceding claims, in which the amino acid(s) are present in composition (A) in a total content ranging from 5% to 20% by weight, preferably ranging from 5% to 15% by weight and more preferentially ranging from 8% to 12% by weight, relative to the total weight of composition (A).

4. The process as claimed in any one of the preceding claims, in which the pH of composition (A) is from 2 to 11, preferably from 4 to 10 and more preferentially from 8 to 10.

5. The process as claimed in any one of the preceding claims, in which composition (A) comprises a total content of coloring agents and/or reducing agents of less than 0.1% by weight, preferably less than 0.01% by weight, more preferentially less than 0.001% by weight relative to the total weight of composition (A); even more preferentially, composition (A) is free of coloring agents and/or reducing agents.

6. The process as claimed in any one of the preceding claims, in which composition (A) comprises at least one organic solvent, preferably chosen from monoalcohols, polyols, polyol ethers and mixtures thereof.

7. The process as claimed in the preceding claim, in which composition (A) comprises a total content of organic solvents ranging from 1% to 40% by weight, preferably ranging from 5% to 30% by weight, more preferentially ranging from 8% to 15% by weight relative to the total weight of composition (A).

8. The process as claimed in any one of the preceding claims, in which the step of permanent reshaping of the keratin fibers comprises the application to the keratin fibers of a composition comprising one or more thiol-based or non-thiol reducing agents, preferably one or more thiol-based reducing agents.

9. The process as claimed in the preceding claim, in which the thiol-based reducing agent(s) are chosen from those of formulae i-1 and i-2, and also the salts thereof, the solvates thereof such as hydrates, and mixtures thereof:
R₁—SH i-1
R′—S—R″ i-2
in which formulae i-1 and i-2:
R₁ represents:
a (C₁-C₈)alkyl and preferably (C₁-C₆)alkyl group, optionally substituted with one or more groups chosen from carboxyl C(O)OH, (di)(C₁-C₄)(alkyl)amino, hydroxyl —OH, thiol —SH or —C(O)—NH—CH₂—C(O)OH and/or optionally interrupted with one or more heteroatoms or groups chosen from —O—, —S—, —N(R′″)—, C(O) or combinations thereof such as —O—C(O)—, —C(O)—O—, —N(R′″)—C(O)—, or —C(O)—N(R′″)—; with R′″ representing a hydrogen atom or a (C₁-C₆)alkyl group; or
a (hetero)aryl group optionally substituted with one or more hydroxyl, thiol or carboxyl groups;
R′ and R″, which may be identical or different, represent a (C₁-C₈)alkyl and preferably (C₁-C₆)alkyl group, substituted with one or more groups chosen from hydroxyl, thiol and carboxyl;
or else R′ and R″ form, together with the sulfur atom which bears them, a 5- to 7-membered heterocyclic group, which is preferably saturated, which comprises from 1 to 3 heteroatoms, and which is optionally substituted with one or more (C₁-C₆)alkyl groups optionally substituted with one or more hydroxyl, thiol or carboxyl groups; more preferentially, the heterocyclic group is a dithiolane group optionally substituted with a (C₁-C₆)alkyl group optionally substituted with one or more carboxyl groups;
the reducing agent(s) preferably being chosen from those of formula i-1 as defined previously;
the reducing agent(s) being more preferentially chosen from those of formula i-1 in which R₁ represents a (C₁-C₈)alkyl and preferably (C₁-C₆)alkyl group,
substituted with one or more groups chosen from carboxyl C(O)OH, amino, hydroxyl —OH and thiol —SH; and/or
optionally interrupted with one or more heteroatoms or groups chosen from —O—, —N(R′″)—, C(O) or combinations thereof such as —O—C(O)—, —C(O)—O—, —N(R′″)—C(O)—, or —C(O)—N(R′″)—, with R′″ representing a hydrogen atom or a (C₁-C₆)alkyl group.

10. The process as claimed in either of claims 8 and 9, in which the thiol-based reducing agent(s) are chosen from thioglycolic acid, thiolactic acid, cysteine, cysteamine, homocysteine, glutathione, thioglycerol, thiomalic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters and amides, notably glyceryl monothioglycolate, and mixtures thereof, preferably from thioglycolic acid, thiolactic acid, cysteamine, and mixtures thereof, more preferentially thioglycolic acid, thiolactic acid, and mixtures thereof.

11. The process as claimed in any one of the preceding claims, in which the pH of the composition comprising the thiol-based reducing agent(s) is acidic, preferably ranging from 1 to 6, more preferentially ranging from 2 to 5 and even more preferentially ranging from 2.5 to 4.

12. The process as claimed in any one of claims 1 to 7, in which the step of permanent reshaping of the keratin fibers comprises the application to the keratin fibers of a composition comprising one or more alkaline agents chosen from mineral hydroxides, organic hydroxides and mixtures thereof, preferably from mineral hydroxides, more preferentially from alkali metal hydroxides, alkaline-earth metal hydroxides, transition metal hydroxides and mixtures thereof, even more preferentially from sodium hydroxide, lithium hydroxide, calcium hydroxide, magnesium hydroxide, barium hydroxide, strontium hydroxide, manganese hydroxide, zinc hydroxide and mixtures thereof; most preferentially, the alkaline agent is sodium hydroxide.

13. The process as claimed in any one of the preceding claims, also comprising, between steps i) and ii), a step i′) consisting in leaving composition (A) to stand on the keratin fibers for a time ranging from 1 min to 60 min, preferably ranging from 3 min to 40 min and more preferentially ranging from 3 min to 20 min.

14. The process as claimed in any one of the preceding claims, also comprising, after step i) or i′) and before step ii), a step i″) of rinsing and/or drying the keratin fibers, preferably a step i″) of drying the keratin fibers.

15. The process as claimed in any one of the preceding claims, in which the process does not comprise a rinsing step i″) between step i) or i′) and step ii).

16. The process as claimed in any one of the preceding claims, also comprising, after step i) or i′) or i″) and before step ii) and/or after step ii), preferably after step i) or i′) or i″) and before step ii), a step of dyeing or bleaching the keratin fibers.

17. The process as claimed in the preceding claim, in which the step of dyeing or bleaching the keratin fibers comprises the application to the keratin fibers of a dyeing or bleaching composition comprising at least one chemical oxidizing agent, preferably chosen from hydrogen peroxide, urea peroxide, alkali metal bromates, peroxygenated salts, peracids and precursors thereof, and mixtures thereof, more preferentially from hydrogen peroxide, peroxygenated salts and mixtures thereof, even more preferentially from hydrogen peroxide, persulfates, perborates or percarbonates of alkali metals or alkaline-earth metals or of ammonium and mixtures thereof, most preferentially hydrogen peroxide.

18. The process as claimed in claim 16 or 17, in which the dyeing step comprises the application to the keratin fibers of a dye composition comprising at least one coloring agent chosen from oxidation dyes, direct dyes and mixtures thereof, preferably from oxidation dyes.

19. A composition (A) as defined in any one of claims 1 to 7.

20. The use of a composition (A) as defined in any one of claims 1 to 7, as a pretreatment composition of a process for permanently reshaping keratin fibers, preferably as a pretreatment composition of a process for straightening or relaxing keratin fibers.

21. The use of a composition (A) as defined in any one of claims 1 to 7, for protecting keratin fibers, preferably for protecting them from breakage, during a treatment for permanently reshaping the keratin fibers.

22. A multi-compartment device comprising:
a first compartment containing a composition (A) as defined in any one of claims 1 to 7; and
a second compartment containing a composition comprising at least one thiol-based or non-thiol reducing agent or at least one alkaline agent chosen from mineral hydroxides, organic hydroxides and mixtures thereof; and
optionally a third compartment containing a composition comprising at least one chemical oxidizing agent; and
optionally a fourth compartment containing a composition comprising at least one coloring agent chosen from oxidation dyes, direct dyes and mixtures thereof.
